(19) United States
(12) Patent Application Publication
(10) Pub. No.: US 2004/0254737 A1
(43) Pub. Date: Dec. 16, 2004

(54) Information processing apparatus, information processing method, storage medium and program

(75) Inventors: Nobuko Yamamoto, Kanagawa (JP); Takashi Tokura, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 10/863,503

(22) Filed: Jun. 9, 2004

(30) Foreign Application Priority Data
Jun. 10, 2003 (JP) 2003-165029

(51) Int. Cl. G01N 33/48; C12Q 1/68; G06F 19/00

(52) U.S. Cl. 702/19; 435/6; 702/20

(57) ABSTRACT

A database capable of accurately analyzing the relation between physical constitution and diseases based on the amount of expression of MHC genes and disease related genes, and extracting useful medical information is efficiently constructed. For solving such a problem, a method according to the present invention for constructing a database for extracting medical information based on MHC gene information of a test object obtained using a DNA chip and disease related gene information includes steps of: acquiring MHC gene information and disease related gene information, and biochemical examination information of the test object and information concerning symptoms (step S105); analyzing the acquired information based on the database (step S107); outputting medical information for the test object obtained by the analysis (step S108); obtaining information concerning drug effects on the test object, and registering the same in the database together with the acquired information; and calculating payback expenses of expenses collected for the output of medical information, which are to be paid back to providers of information previously registered in the database.

### FIELD OF THE INVENTION

The present invention relates to construction of a database storing information for extracting various kinds of medical information such as information concerning expression of genes associated with physical constitution and disease associated genes existing on chromosome genomes, analysis of correlation between symptoms and clinical test data, disease morbidity risks and drugs to be administered.

### BACKGROUND OF THE INVENTION

A human genome is composed of three billions of base pairs and widely varies depending on individuals. It is believed that this variety is ascribable to the fact that very rare errors in DNA replication occurring in reproductive cells during chromosome replication have been accumulated and inherited since human beings came into the world. This variety is classified into changes (variations) resulting from base replacement, insertion or deficiency, and the change occurs over only one base, over several bases or over a considerable length.

Some of changes in genes, alone or in combination of two or more changes, appear as some individual difference, for example a difference in biological function such as a difference in physical constitution. The difference in biological function refers to, for example, a difference in onset risk for a specific disease and a difference in side effect for a specific drug, and it is believed that the change in genes has a close relation with these differences.

In fact, it has become evident that these changes (variations, variants or polytypes) in genes are related closely to onsets and onset risks of disease such as diabetes, fatness, hypertension, arterial sclerosis, cancers, hyperlipemia, cardiac disease, immune allergy, asthma, osteopathy, neuropathy and Alzheimer. Generally, if a change in a single gene is associated directly with the onset as in genetic disease, the gene is called a disease causing gene, and if a plurality of genetic grounds are involved, and changes in individual genes increase the disease onset risk, the gene is called a disease sensitive gene. Useful medical information can be provided based on expression of the gene.

As one example, Japanese Patent Laid-Open No. 2001-112486 discloses a technique in which analysis is performed on existence/nonexistence of changes in base sequences of genes, and screening information and analysis results specific to the genes are recorded in a portable recording medium by an optical method.

Further, the publication makes references to numerous diseases associated with the change in the gene are mentioned, and discloses a technique for systematically and automatically utilizing medical information such as disease morbidity risks and responsiveness and side effects of drugs by detecting changes in genes.

Furthermore, Japanese Patent Laid-Open No. 2001-112486 discloses a technique in which results of examining changes in autosomes are classified into the normal type (W) and the abnormal type (m), an evaluation is made on a pair of chromosomes with three types of combinations of (W/W), (m/W) and (m/m), and medical information such as disease onset risks and drug sensitivity is provided from correlation between genes determined to be abnormal and related diseases.

However, as shown in the prior art described above, it is prerequisite that mutually correlated gene data is sufficiently accumulated in advance for detecting changes in genes to obtain useful medical information such as disease morbidity risks and responsiveness and sensitivity of drugs. However, since it is estimated that diseases such as life habit diseases each involve about 10 to 20 disease sensitive genes, and it is believed that several types of gene changes (variations, variants and polytypes) exist for each disease sensitive gene, considerable time will be required for elucidating the changes and clarifying relations between those changes and changes in genes representing physical constitution and the like.

Furthermore, information on SNPs is quite numerous, and still more time will be required for collecting and analyzing the information to correlate the information with physical constitution. Thus, even if studies are conducted on presumed items of SNPs, more information is newly discovered, and the studies are left insufficient for ever.

Then, as means for avoiding the problem, a technique using major histocompatibility complex genes (MHC) has received attention in recent years. The MHC gene is a region in which immune genes are most concentrated in a human genome, and has recently received attention with its base sequence clarified (Nature volume 401, p921-923, 1999).

The MHC genes (HLA antigens for human beings) include three types: HLA-A, B and C as class I antigens, and three types: HLA-DR, DQ and DP as class II antigens, and genes exist in correspondence therewith. Each individual is given each type of antigen from each of parents, that is, total 12 types of antigens, which form a “type” of the individual. That is, by examining MHC genes, a difference in biological function between individuals can be obtained.

Hitherto, about 1000 types of genes of HLA-A, HLA-B, HLA-C, HLA-DR, HLA-DQ and HLA-DP together have been identified, and it is expected that new MHC genes will be elucidated and the number of identified MHC genes will increase in future.

Case examples of medical applications of such MHC genes are emerging. For example, genes involved in judgment on compatibility/incompatibility in bone marrow transplantation, organ transplantation and the like exist in sequences of MHC genes, and judgment on compatibility/incompatibility in organ transplantation and bone marrow transplantation by examination using leukocyte is presumed to be replaced by typing with MHC genes in the future.

Further, recently, the relation between MHC genes and disease has been clarified. For example, HLA-DRB1*0401 is considered as being correlated with the onset of chronic rheumatism (J. Clin. Invest., 89:2033, 1992), and it is shown that the insulin autoimmune syndrome has HLA-DRB1*0406. In addition thereto, there are many diseases showing strong correlations with HLA antigens and among them, famous diseases include juvenile onset diabetes, psoriasis vulgaris and Behcet disease.

It is conceivable that elucidation of correlation between MHC physical constitution and diseases utilizing MHC genes will progress in the future. Accordingly, a database suitable for elucidation of correlation between physical constitution and the amount of expression of disease related genes utilizing MHC genes is strongly desired.

### SUMMARY OF THE INVENTION

The present invention has been made in view of the above problems, and its object is to provide an information processing apparatus, an information processing method, a program and a storage medium for efficient construction of a database capable of accurately analyzing a relation between physical constitution and a disease based on MHC gene and disease related gene information, and extracting useful medical information.

For achieving the above object, the information processing apparatus according to the present invention has the following configuration. Specifically, it is an information processing apparatus constructing a database for extracting predetermined medical information based on information concerning a major histocompatibility complex (MHC) of a test object obtained using a DNA chip and disease related gene information, comprising: acquiring unit configured to acquire the information concerning the major histocompatibility complex and disease related gene information, and biochemical examination information of the test object and information concerning symptoms; analyzing unit configured to analyze the information acquired by the acquiring unit based on the database; outputting unit configured to output medical information for the test object obtained by the analyzing unit; registering unit configured to acquire information concerning drug effects on the test object, and registering the information together with the information acquired by the acquiring unit; and calculating unit configured to calculate payback expenses of expenses collected for output of the medical information, which are to be paid back to providers of information previously registered in the database.

According to the present invention, a database capable of accurately analyzing a correlation between physical constitution and a disease based on the amounts of expression of MHC genes and disease related genes and extracting useful medical information can be efficiently constructed.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 shows a flow of overall processing including an information processing method according to one embodiment of the present invention;

FIG. 2 shows one example of information (information concerning MHC genes and disease related genes) inputted by an information provider;

FIG. 3 shows one example of information registered in a database;

FIG. 4 shows one example of information (biochemical examination information) inputted by the information provider;

FIG. 5 shows one example of analysis results shown to the information provider;

FIG. 6 shows one example of information (information concerning drug effects) inputted by the information provider;

FIG. 7 shows a flow of analysis procedure processing in an information processing apparatus according to the first embodiment of the present invention;

FIG. 8 shows a flow of payback expense calculation processing in the information processing apparatus according to the first embodiment of the present invention; and

FIG. 9 shows the configuration of the information processing apparatus according to the first embodiment of the present invention.

### DETAILED DESCRIPTION OF THE PREFERRED EMBODIMENTS

Preferred embodiments of the present invention will now be described in detail in accordance with the accompanying drawings.

An outline of a business model (one example) for construction of a database realized using an information processing apparatus according to the present invention will be briefly described.

[Outline of Business Model]

By using the information processing apparatus, the following business model can be assumed. First, a doctor purchases a DNA chip having physical constitution related genes (genome typing) arranged thereon and a DNA chip having disease related genes arranged thereon from a database manager. Then, the doctor uses the purchased DNA chips to examine a specimen from a patient, accesses a Web site of the database manager on the internet, and inputs information such as the MHC gene data and disease related gene data (collectively referred to as “gene data”), the symptom of the patient, and clinical test data (biochemical examination information such as urine examination, feces examination items and blood examination). For the inputted information, the database manager extracts information (analysis results) concerning possible disease candidates, clinical test items to be obtained as additional data and drugs to be administered. The doctor acquires the information via the internet and pays an information fee for the information.

That is, the database manager provides appropriate DNA chips, manages MHC gene data and disease related gene information by the DNA chips and the like, and provides various analysis results as a business. For this purpose, the database manager constructs analysis software having physical constitution, clinical test data and disease related genes correlated to one another using known information as a basis, and also prepares information concerning disease candidates and drugs to be administered. Further, the database manager analyzes MHC gene data and disease related gene information and the like provided via the internet, and presents information concerning names of disease, clinical test items to be added and drugs to be administered to the information provider (doctor in this case). At this time, the database manager demands a usage fee from the information provider. Further, the provided MHC gene data and disease related gene information and the like are added to the database as additional data, and the accuracy of the analysis software is improved or new items are added, whereby the function of the analysis software is expanded.

Further, the database manager has a right to use MHC gene data and the like provided by the information provider and once added to the database in response to a request from other person. If as a result, the other person accesses the Web site and acquires analysis results obtained by accessing the database including MHC gene data and the like provided by the information provider for predetermined disease items, part of the usage fee paid to the database manager from the other person is paid as a payback fee to the information provider who provided MHC gene data and the like to the database. The payback fee paid at this time should be consistent with the amount of information contributing to analysis of the disease.

Furthermore, the database manager has a right to use all data, and has a right to use the data for every application such as development of software for analyzing the relation between genes related to physical constitution such as fatness and genomes, and the like, in addition to development of analysis software having physical constitution, clinical test data and disease related genes correlated with one another.

By establishing such a model, a desired database can be efficiently constructed.

[Flow of Overall Processing]

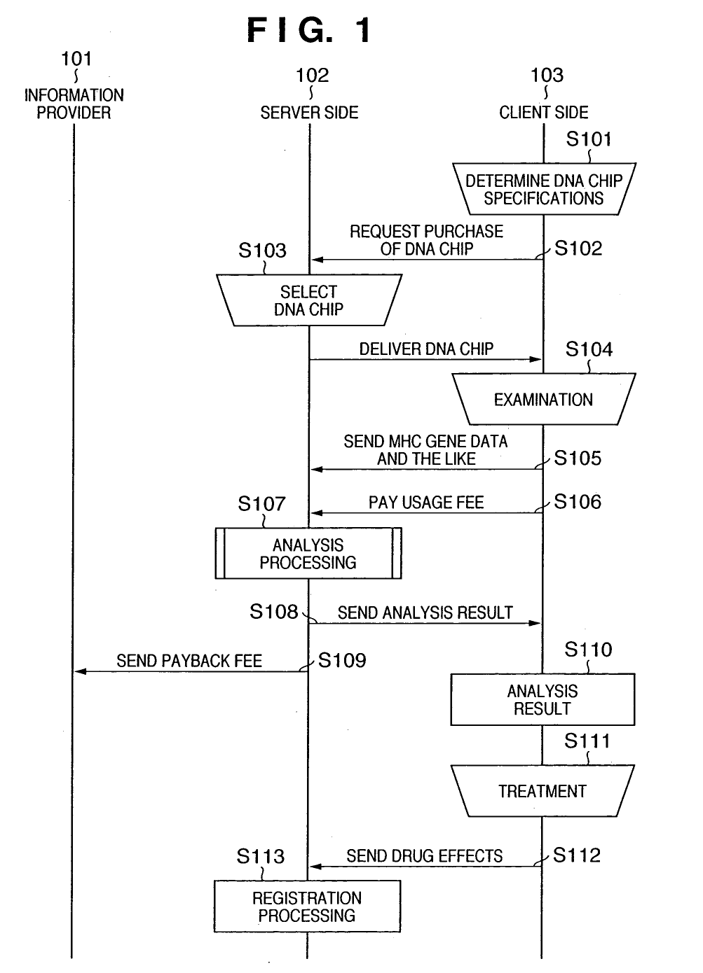

The flow of processing in the business model described above will now be described more specifically using FIG. 1. In FIG. 1, operations by a doctor or processing at a client terminal possessed by the doctor are described on the client side (103). Furthermore, operations by a database manager or processing at a server terminal in which a database is stored are described on the server side (102). Furthermore, an information provider 101 is a person who provided MHC gene data and the like to the server side and registered the data in the database of the server in the past.

At step S101, items to be examined such as physical constitution of a patient and disease related genes are organized and specifications of a DNA chip to be purchased are determined on the client side (103). Furthermore, in this embodiment, major histocompatibility complex (MHC) genes are used as genes related to physical constitution (genome typing). This does not exclude use of SNIPs as genes related to physical constitution. Furthermore, items to be examined, of disease related genes, are concerned with, for example, hypertension, diabetes, cardiac disease, arterial sclerosis, hyperlipemia, allergic disease, asthma and the like for internal medicine, neuropathy, Alzheimer, schizophrenia, melancholia and the like for psychopathy, and arthropathy, chronic rheumatism and the like for orthopaedics. However, the items are not limited thereto as disease sensitive genes. Information concerning determined DNA chip specifications is sent to the server side 102 together with DNA chip purchase request information (step S102).

At step S103, an optimum DNA chip is selected based on the information concerning the DNA specifications on the server side (102). The selected DNA chip is delivered to the client side (103) in exchange for payment. In this way, the client side (103) may purchase a DNA chip for examining genes of MHC genes related to physical constitution and genes involved in a disease in each area of expertise from the server side. MHC genes and disease related genes may exist on the same chip, or they may exist on separate ships.

At step S104, the sent DNA chip is used to examine physical constitution related genes of a patient (donor) and obtain a hybrid patter on the client side (103). The MHC type can be determined based on the hybrid pattern, and it can be registered, but such a step is not prerequisite.

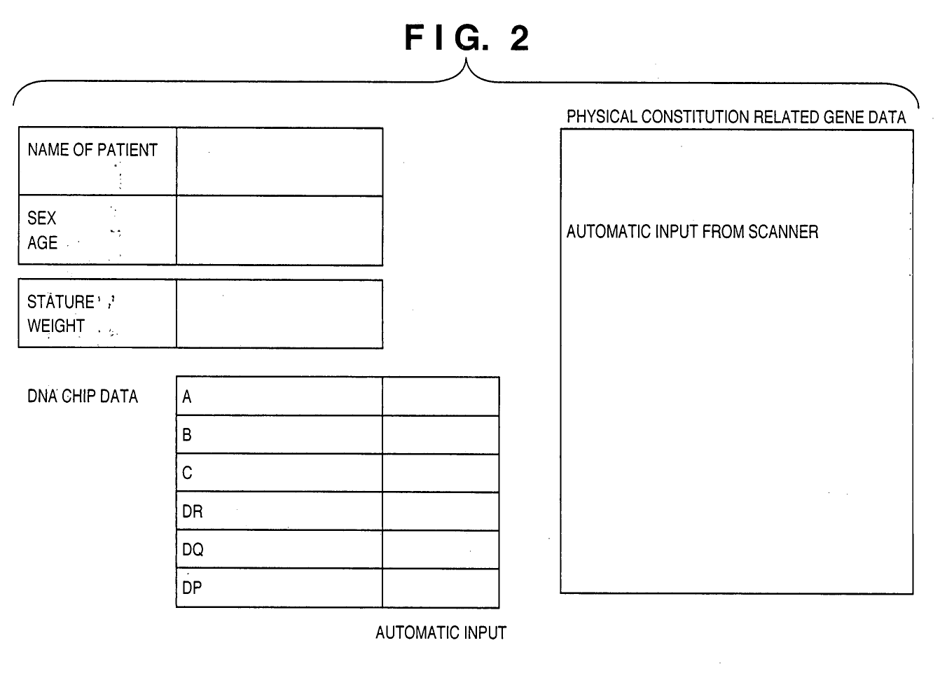

On the client side (103), the hybrid pattern is preferably inputted directly as data, but data directly acquired through a scanner suitable for the DNA chip provided by the database manager may be registered. FIG. 2 shows one example of a format in which physical constitution related gene data directly acquired through the scanner is described. In this way, the scanner capable of directly performing registration operations as physical constitution related gene data can more likely prevent determination errors caused by reading misses.

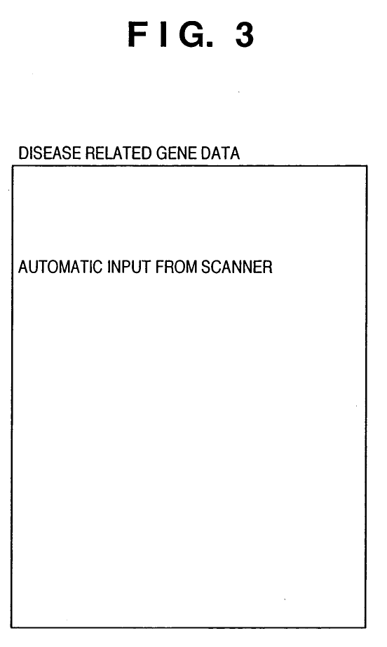

Similarly, the sent DNA chip is used to examine disease related genes of the patient on the client side (103). FIG. 3 shows a format in which disease related gene data directly acquired through the scanner is described.

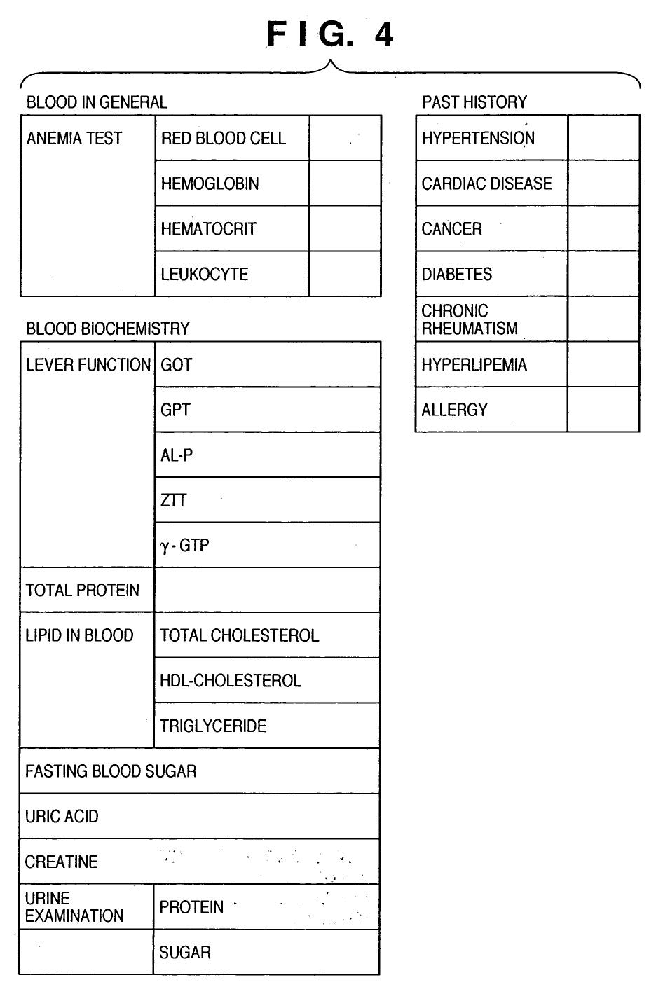

Registration of such gene data (physical constitution related gene data and disease related gene data) is performed by inputting the data on the Web site provided by the server side (102) using the internet (step S105). At the time of inputting the data, a Web usage fee is paid to the database manager (step S106). Furthermore, on the client side (103), information such as other clinical test data and the symptom of the patient is inputted in addition to such gene data. For reference purposes, one example of a format for inputting information such as other clinical test data and the symptom of the patient is shown in FIG. 4.

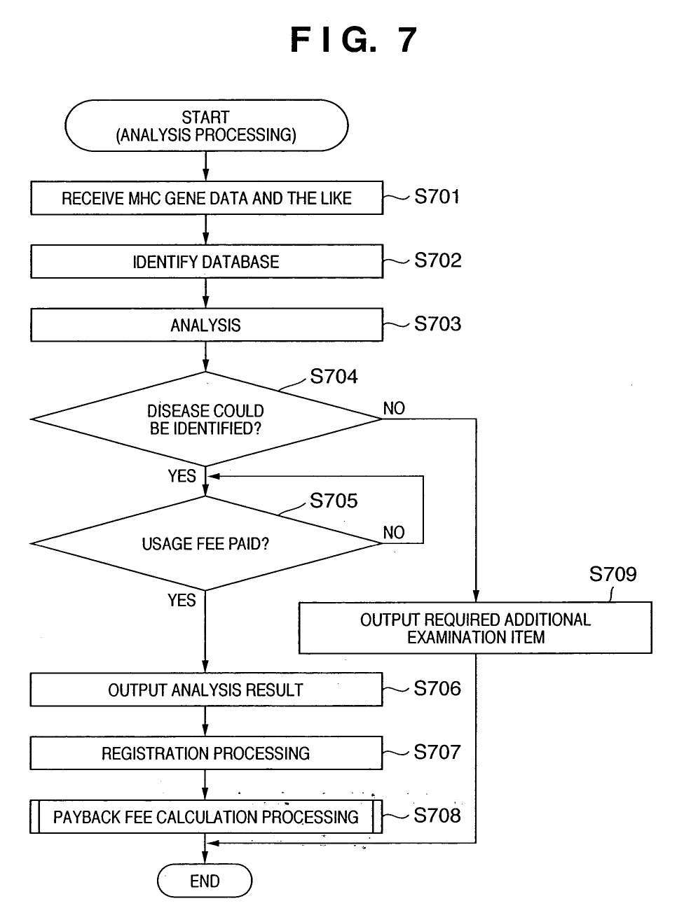

On the server side (102), the stored database is used to automatically perform processing by analysis software to diagnose the disease based on various inputted information (physical constitution related gene data, disease related gene data, clinical test data, the symptom of the patient, etc.) (step S107. It will be described in detail using FIG. 7). The database that is used here has the relation between physical constitutions and diseases stored by the database manager based on known information.

If as a result, a name of disease is identified, a name of a drug effective for the disease, identified in consideration of the relation between the name of disease and the physical constitution of the patient is sent as an analysis result (step S108).

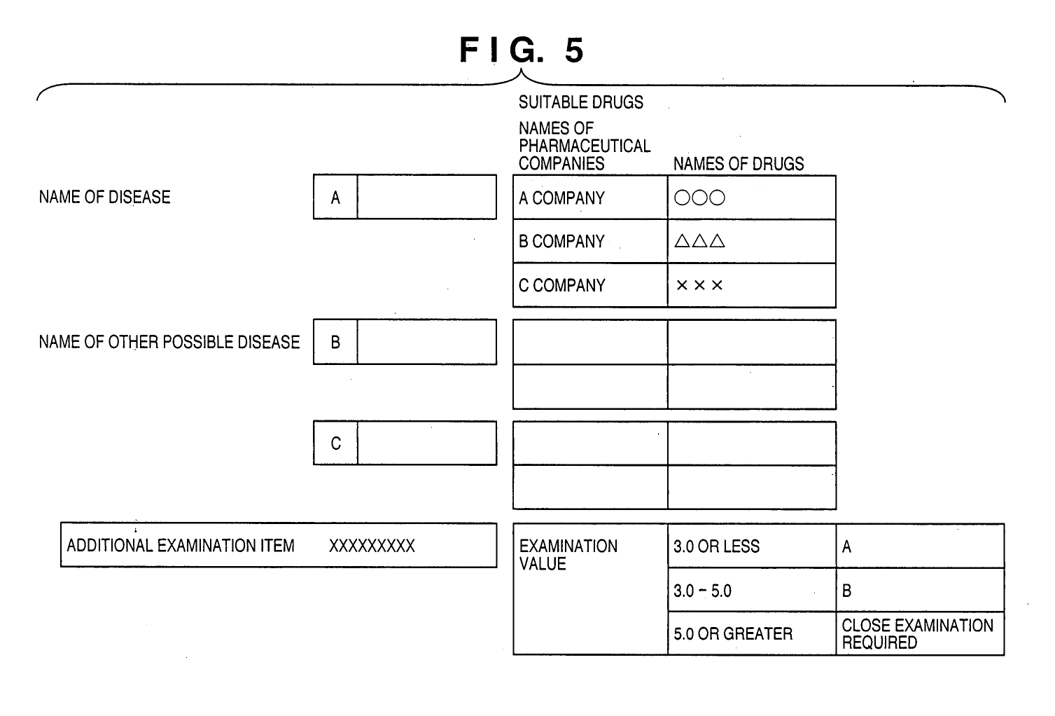

On the other hand, if a name of disease is not identified, a type of clinical test data further required is sent to the client side (103) as an analysis result (step S108). Furthermore, the possibility of disease for determination on the negative/positive if the required test is conducted. For reference purposes, one example of a format in which the analysis result is described is shown in FIG. 5. Furthermore, if a name of disease is identified, the analysis result is additionally registered in the database.

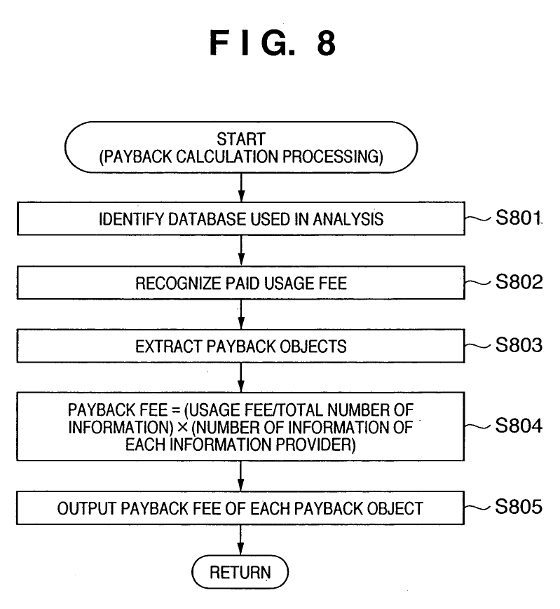

On the other hand, a payback fee is sent to the information provider (101) who has already registered gene data and the like in the database used in analysis processing at step S107 (step S109. It will be described in detail using FIG. 8).

Furthermore, the analysis result sent at step S108 is displayed on the client side (103) (step S110) and on the client side (103), various kinds of treatments are performed based on the analysis result (step S111).

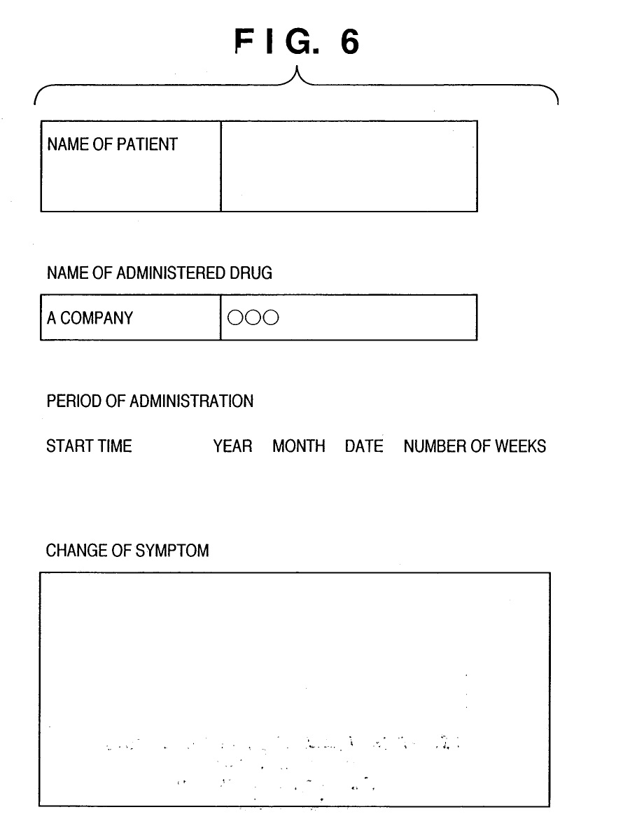

Further, at step S112, the result of giving a drug based on the provided analysis result (drug effect, see FIG. 6) is sent to the server side, and additionally registered in the database (step S113).

[Analysis Processing]

Details of analysis processing (step S107) will now be described using FIG. 7. FIG. 2 shows a flow of analysis processing in an information processing apparatus according to the first embodiment of the present invention.

At step S701, information such as gene data sent from the client side (103) is received. At step S702, of databases classified and registered for each item to be examined (hypertension, diabetes, etc.), a database corresponding to information received at step 701 is identified.

At step S703, the identified database is read, and analysis is performed using analysis software. If as a result of analysis, a disease can be identified (“YES” at step S704), processing proceeds to step S705, where whether a predetermined usage fee has been already paid or not is determined (step S705).

If the predetermined usage fee has been already paid, processing proceeds to step S706, where the analysis result is outputted, and the gene data and the like are registered in the database at step S707. Further, at step S708, payback fee calculation processing is carried out.

On the other hand, if a disease cannot be identified (“NO” at step S704), processing proceeds to step S709, where information of which additional examination item is required for identification of a disease is outputted.

[Payback Fee Calculation Processing]

Details of payback fee calculation processing (step S708) will now be described using FIG. 8. The database (database for hypertension, database for diabetes, etc.) used in analysis at step S703 is identified at step S801, and the paid usage fee is recognized at step S802.

At step S803, an information provider who registered gene data and the like in the identified database is extracted. The extracted information provider is the person to whom the payback fee is sent.

At step S804, the payback fee of each information provider is calculated. Specifically, the paid usage fee divided by the total number of information in the database is multiplied by the number of information by each information provider in the database. At step S805, the calculated payback fee for each information provider is outputted.

Thus, as the number of information providers increases, the amount of information increases and the accuracy of analysis is improved, but an amount of money paid to each information provider decreases.

[Example of Use of Database]

An example of use of the database constructed as described above will now be described. Data accumulated in the database is analyzed by analysis software capable of correlating the disease with the physical constitution (MHC type), the age, the sex, and the symptom and clinical test data of each patient aside from a file for each disease. As a result, even for the same symptom and disease, a classification can be made into several types based on the physical constitution and the test value of each clinical test item to analyze the relation between the type and the drug effect.

In this way, a treatment/administration matching each MHC type is accurately estimated, thus making it possible to provide a high level of service to a database user. Furthermore, determination items for diagnosis exist in the database as a flowchart, and therefore even a doctor with less experience can make a diagnosis with the reduced possibility of misdiagnosis.

Furthermore, as a result of performing the analysis described above, the database manager can identify a new disease, which has been difficult to identify, by giving attention to each case where classification is impossible. For the case where misdiagnosis occurred because of similar symptoms, the possibility of other disease can be extracted. Information obtained from analysis of correlation between these data can be added to the database and provided to the information provider as information.

Further, the database manager can also analyze data of the fluorescence amount obtained as a result of a hybridization reaction of a DNA chip. For example, by extracting a difference in the same type of DNA chips between clients, a difference in specimen treatment or a difference by the type of scanner can be extracted and corrected. Alternatively, a method for obtaining reliable data can be presented on the basis of such information. Furthermore, by analyzing data of the same client, the apparatus can be calibrated and errors of the method can be pointed out.

The fluorescence amount of hybrid body by a set of a probe and a specimen fully matching each other varies depending on each sequence. By storing relations between fluorescence amounts and sequences in a database, software for extracting parameters for predicting a fluorescence amount from a sequence is developed. In addition to stability of the hybrid body, addition of such information (for example, adjustment of the amount of probes bound to a substrate during fabrication of the DNA chip) can equalize fluorescence amounts of respective spots and improve the determination accuracy of the DNA chip.

Further, by analyzing the fluorescence amount of a hybrid body entailing a mismatch, a parameter concerning how the stability (and fluorescence amount) of the hybrid body varies depending on the position and type of single base mismatch can be extracted. For typing of the MHC gene, correct identification of a hybrid body having a single base mismatch is an important challenge. It is very effective in probe design to clarify the relation between the sequence and the fluorescence amount of the hybrid body using the above database, and the relation can be fed back for development of a reliable DNA chip.

[Configuration of Information Processing Apparatus (Server Terminal)]

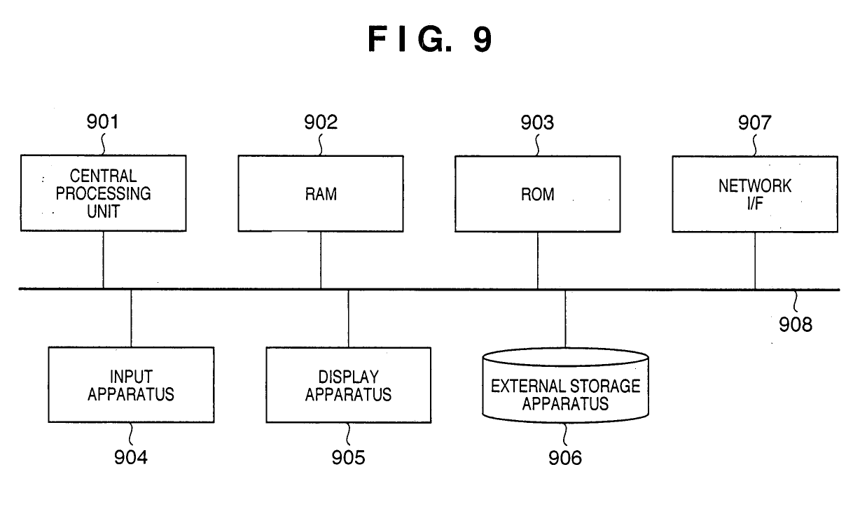

The configuration of an information processing apparatus (server) carrying out the above analysis processing (step S107) and registration processing (step S113) is shown in FIG. 9.

In FIG. 9, reference numeral 903 denotes a control memory (ROM), reference numeral 901 denotes a central processing unit, reference numeral 902 denotes a memory (RAM), reference numeral 906 denotes an external storage apparatus, reference numeral 907 denotes a network I/F, reference numeral 904 denotes an input apparatus, reference numeral 905 denotes a display apparatus, and reference numeral 908 denotes a bus. A control program for realizing the analysis processing (step S107) and registration processing (step S113) according to this embodiment, and data for use in the control program are stored in the ROM 903. The control program and data are captured in the memory 902 as appropriate through the bus 908 under control by the central processing unit 901, and executed by the central processing unit 901. Furthermore, a constructed database is stored in the external storage apparatus 906.

As apparent from the above description, according to this embodiment, a doctor can upload information of the symptom of a patient and the like, MHC gene data and disease related gene data, and obtain various kinds of medical information of a disease onset risk, drug sensitivity and the like in exchange for the uploaded data.

On the other hand, at a server terminal, the uploaded data is accumulated in the database, and analyzed by a statistical process, and a relation between the MHC type and the disease and patient's symptom is outputted. Further, data concerning the effect of a drug administered based on the analysis result is added to the database.

In this way, according to this embodiment, a database capable of extracting a relation between physical constitution and a disease based on the amount of expression of MHC genes and disease related genes, and extracting useful medical information can be efficiently constructed.

[Second Embodiment]

Furthermore, it is desirable that MHC gene data and disease related gene data should be inputted to the database constructed on the server side (102) via a DNA array dedicated scanner for reducing a work load during input.

At this time, the scanner is provided with a form for inputting the name, sex, age, symptom and the like of a patient, and a doctor can manage various kinds of information as chart information.

Furthermore, for clinical test data, it is preferable that if it is possible to make a link to a testing department of a hospital or a clinical test company via a scanner, data of both sides are automatically inputted. Furthermore, a mechanism is established such that such information is all subjected to encryption processing (processing for replacement with sample ID) when connection is established with the database, and thus patient information is never registered in the database as personal information.

[Other Embodiments]

Furthermore, the present invention may be applied to a system comprised of a plurality of devices (for example, host computer, interface device, reader, printer, etc.), or may be applied to an apparatus comprised of one device (for example, copier, facsimile apparatus, etc.).

Furthermore, the object of the present invention is also achieved by supplying to a system or apparatus a storage medium having recorded therein a program code of software for realizing the function of the embodiment described above, and making a computer (or CPU or MPU) of the system or apparatus read the program code stored in the storage medium, as a matter of course.

In this case, the program code itself read from the storage medium achieves the function of the embodiment described above, and the storage medium having the program code recorded therein constitutes the present invention.

For the storage medium for supplying the program code, for example, a floppy® disk, hard disk, optical disk, magneto-optical disk, CD-ROM, CD-R, magnetic tape, nonvolatile memory card, ROM or the like may be used.

Furthermore, the present invention includes not only the case where the function of the embodiment described above is realized by executing the program code read by the computer, but also the case where the OS (operation system) or the like operating on the computer performs part or all of actual processing based on instructions of the program code, whereby the function of the embodiment described above is realized, as a matter of course.

Further, the present invention also includes the case where the program code read from the storage medium is written in a memory provided in a feature expansion board inserted in the computer or a feature expansion unit connected to the computer, and then a CPU or the like provided in the feature expansion board or feature expansion unit performs part or all of actual processing based on instructions of the program code, whereby the function of the embodiment described above is realized, as a matter of course.

The present invention is not limited to the above embodiments and various changes and modifications can be made within the spirit and scope of the present invention. Therefore to apprise the public of the scope of the present invention, the following claims are made.

What is claimed is:

1. An information processing apparatus constructing a database for extracting predetermined medical information based on information concerning a major histocompatibility complex of a test object obtained using a DNA chip and disease related gene information, comprising:
acquiring unit configured to acquire the information concerning the major histocompatibility complex and disease related gene information, and biochemical examination information of the test object and information concerning symptoms;
analyzing unit configured to analyze the information acquired by said acquiring unit based on said database;
outputting unit configured to output medical information for the test object obtained by said analyzing unit;
registering unit configured to acquire information concerning drug effects on the test object, and registering the information together with the information acquired by said acquiring unit; and
calculating unit configured to calculate payback expenses of expenses collected for output of the medical information,.which are to be paid back to providers of information previously registered in the database.

2. The information processing apparatus according to claim 1, wherein probes of major histocompatibility complex genes bound to the DNA chip are a probe set for determining the type of HLA antigen.

3. The information processing apparatus according to claim 1, wherein the medical information for the test object includes information of disease names and effective drugs and disease related gene information.

4. The information processing apparatus according to claim 2, wherein the information concerning the major histocompatibility complex is data obtained by reading by an image reading apparatus an examination result obtained using the DNA chip.

5. The information processing apparatus according to claim 1, wherein the biochemical examination information includes clinical test data concerning blood and urine collected from the test object.

6. The information processing apparatus according to claim 1, wherein said acquiring unit acquires the information concerning the major histocompatibility complex and disease related gene information, and biochemical examination information of the test object and information concerning symptoms via the internet.

7. The information processing apparatus according to claim 1, wherein said analyzing unit analyzes physical constitution of the test object based on the information concerning the major histocompatibility complex of the test object obtained using the DNA chip, and analyzes an expression state of disease related genes based on the disease related gene information.

8. The information processing apparatus according to claim 1, wherein said registering unit encodes the information acquired by said acquiring unit, and replaces the information concerning the test object with a sample ID and registers the same in the database.

9. The information processing apparatus according to claim 1, wherein said calculating unit calculates the payback expense for each provider according to the amount of information for each provider registered in the database.

10. An information processing method for constructing a database for extracting predetermined medical information based on information concerning a major histocompatibility complex of a test object obtained using a DNA chip and disease related gene information, comprising:
an acquiring step of acquiring the information concerning the major histocompatibility complex and disease related gene information, and biochemical examination information of the test object and information concerning symptoms;
an analyzing step of analyzing the information acquired by said acquiring step based on the database;
an outputting step of outputting medical information for the test object obtained by said analyzing step;
a registering step of acquiring information concerning drug effects on the test object, and registering the information together with the information acquired by said acquiring step; and
a calculating step of calculating payback expenses of expenses collected for output of the medical information, which are to be paid back to providers of information previously registered in the database.

11. The information processing method according to claim 10, wherein the DNA chip is obtained by binding a probe set for determining the type of HLA antigen, of base sequences of major histocompatibility complex genes.

12. The information processing method according to claim 10, wherein the medical information for the test object includes information of disease names and effective drugs.

13. The information processing method according to claim 11, wherein the information concerning the major histocompatibility complex and disease related gene information are data obtained by reading by an image reading apparatus an examination result obtained using the DNA chip.

14. The information processing method according to claim 10, wherein the biochemical examination information includes clinical test data concerning blood and urine collected from the test object.

15. The information processing method according to claim 10, wherein in said acquiring step, the information concerning the major histocompatibility complex and disease related gene information, and biochemical examination information of the test object and information concerning symptoms is acquired via the internet.

16. The information processing method according to claim 10, wherein in said analyzing step, the physical constitution of the test object is analyzed based on the information concerning the major histocompatibility complex of the test object obtained using the DNA chip and the disease related gene information.

17. The information processing method according to claim 10, wherein in said registering step, the information acquired by said acquiring step is encoded, and the information concerning the test object is replaced with a sample ID and registered in the database.

18. The information processing method according to claim 10, wherein in said calculating step, the payback expense is calculated for each provider according to the amount of information for each provider registered in the database.

19. A storage medium storing a control program for realizing the information processing method according to claim 10 by a computer.

20. A control program for realizing the information processing method according to claim 10 by a computer.
